(19) United States
(12) Patent Application Publication
(10) Pub. No.: US 2022/0047853 A1
(43) Pub. Date: Feb. 17, 2022

(54) MODULAR CANNULA DEVICE

(71) Applicant: Universitaet des Saarlandes, Saarbruecken (DE)

(72) Inventor: Philipp Lepper, Homburg (DE)

(21) Appl. No.: 17/276,709

(22) PCT Filed: Sep. 16, 2019
(86) PCT No.: PCT/EP2019/074604

§ 371 Date: Mar. 16, 2021

(30) Foreign Application Priority Data
Sep. 17, 2018 (DE) 10 2018 122 616.0

(51) Int. Cl. A61M 25/06; A61M 25/00; A61M 25/01

(52) U.S. Cl. CPC A61M 25/0662; A61M 2025/0006; A61M 25/0108; A61M 25/007

(57) ABSTRACT

In various exemplary embodiments, a modular cannula device is provided that has an external, tubular cannula, a first tubular guide part which has a tapering distal end and can be inserted into the cannula so that its outer wall rests tightly against the inner wall of the cannula, and a second tubular guide part, which has a tapering, pointed distal end and can be inserted into the first guide part so that its outer wall rests tightly against the inner wall of the first guide part.

The invention relates to a modular cannula device which is particularly suitable for use as a right ventricular assist device.

Right ventricular failure is a common problem in patients with severe respiratory failure, in patients with an implanted left ventricular assist device (LVAD), and generally in intensive-care patients. Surgically implanted systems have the disadvantage that they require two sternotomy operations—one operation for implantation and another operation to remove the system. In the case of patients who are already severely ill, these complex and prolonged surgeries contribute considerably to morbidity and mortality. Hitherto, there have only been a few percutaneous systems for hemodynamically relieving the heart, in particular the right ventricle of the heart. These are usually not very efficacious, and implantation is complex.

For the insertion of a percutaneous system which is intended to support cardiac function, the patient does not need to undergo an in-patient operation since these systems are introduced into the body of the patient by the placement of a catheter. This surgery, which is usually performed using the Seldinger technique, requires only a little effort. The placement of the catheter begins using conventional methods by puncturing a large blood vessel with a hollow needle. For percutaneous cardiac support systems, in most cases the upper jugular vein is used for this purpose, since it allows a quite short and predominantly linear access to the heart. A guide wire is then inserted into the jugular vein via a hollow needle and advanced to just before the right atrium. A sheath is then inserted along the guide wire, and the guide wire is removed again. A balloon catheter can be advanced through the placed sheath to its target location, the pulmonary artery. A second, relatively thin wire is then pushed through the single-lumen balloon catheter. The balloon catheter, which has been used to place the second wire “around the curve” i.e., from the atrium into the pulmonary artery, is then removed, and a switch is then made to a so-called pigtail catheter via the placed second wire. A stiffer third wire is then placed through the pigtail catheter. The sheath can then be removed to finally allow the cannula last employed to be advanced along the wire into the heart and pulmonary artery. In this case, the stiffer third wire serves as a guide rail which forces the finally inserted cannula within the right ventricle along a U-shaped path.

In the event of an error, for example should the balloon catheter or the guide wire slip, the procedure must be restarted practically from the beginning since the balloon catheter, which created the necessary dilation to provide the U-shaped curvature necessary for passing through the ventricle, is no longer placed. Slippage of the balloon catheter and/or the guide wire is facilitated by the fact that the balloon catheter, which usually has a length of 1.50 m, can be moved relative to the guide wire, which is significantly longer with a length of up to 2.60 m.

Given the above description of the usual method steps in placement of a catheter, it is apparent that the entire operation is associated with numerous processes of inserting and removing objects (guide wire, catheter) into and out of the utilized vein, each of which takes a certain amount of time and can lead to ablation of tissue from the inner wall of the vessel concerned. In addition, any insertion of an object, such as the wire or a catheter, into the corresponding blood vessel entails the risk of perforating the vessel wall, in particular in older patients or those whose blood vessels are weakened as a result of illness. Moreover, the numerous change-overs of catheters and wires promotes incorrect positioning, which in the worst case means that the procedure must be restarted from the beginning. Although the method is nowadays well mastered by specialized physicians, it is apparent that it nevertheless poses a certain overall risk to the patient.

In light of this problem, the present invention provides a modular cannula device which, in terms of handling, can facilitate the process of placing a cannula by the tip into the pulmonary artery, particularly placing an arterial ECMO cannula (ECMO: extracorporeal membrane oxygenation), and at the same time can significantly shorten the operation time.

In various embodiments, a modular cannula device is provided which has an external, tubular cannula. Furthermore, the modular cannula device has a first tubular guide part that has a tapering distal end and can be inserted into the external cannula so that its outer wall rests tightly against the inner wall of the cannula. Furthermore, the modular cannula device has a second tubular guide part which has a tapering, pointed distal end and can be inserted into the first guide part so that its outer wall rests tightly against the inner wall of the first guide part. The external cannula may take the form of a tube or sleeve having a length within a range of, for example, 50 cm to 3 m. Likewise, the guide parts may be tubular or sleeve-like with a corresponding dimension matching the dimension of the cannula so that in each case, an inner module of the modular cannula device can be inserted into the module that is correspondingly external relative thereto. Not counting the distal regions of the two guide parts, their lengths can correspond at least to the length of the cannula or can even be longer. Lateral holes may be provided in the cover or in the side wall of the distal end region of the cannula.

The external cannula of the cannula device according to the invention is preferably the cannula to be inserted finally, for example an arterial ECMO cannula. A two-part guide part adapted thereto can be inserted into this final cannula. On the one hand, the two-part guide part ensures that the hollow space in the external cannula remains closed during the placement of the modular cannula device and blood thus cannot pass therethrough so that patient blood loss is prevented. On the other hand, the two-part guide part can be designed to be stiffer than the external cannula and serve as a support part that stiffens the cannula in order to allow the insertion of cannulas made of soft/flexible material into the body. Consequently, by applying the external cannula to the two-part guide part, cannulas can be introduced into the body that would otherwise buckle or collapse during the insertion process and which could not therefore be guided through vessels of the body at all, or only with great effort.

The first guide part which can be inserted into the external cannula can correspond to an external guide of a two-layer stylet, and the second guide part which can also be inserted thereinto can correspond to an inner guide of the two-layer stylet. The tapering end of the first and second guide parts may narrow sharply or conically. In the assembled state (i.e., in a state in which the second guide part is inserted into the first guide part), the ends of the two guide parts can transition gradually into one another and form an overall tapered or sharply or conically narrowing distal end of the (two-part) guide part.

In the assembled state of the modular cannula device according to the invention, the outer wall of each module located in the interior of another module lies tightly against the inner wall of the module located outside relative thereto.

According to other embodiments of the cannula device, the opening angle of the tapering distal end of the first guide part may be greater than the opening angle of the tapering distal end of the second guide part. The distal end of the second guide part can thus narrow more sharply than the distal end of the first guide part. Overall, the specific configuration of the opening angles as well as the external shape of the two distal ends can be adapted as required, wherein the adaptation of these parameters as well as the sizes of the cannula and the guide parts can make their adaptation to the size of the patient. The distal end or end region of the cannula device (or one of its modules) according to the invention is understood to mean the end or the end region with which the cannula device is inserted into the patient. The proximal end or end region of the cannula device (or one of its modules) according to the invention is correspondingly understood to be the opposite end or end region.

According to other embodiments of the cannula device, the first tubular guide part may have an enlarged region at the proximal end or in the proximal end region with an outer diameter that is greater than the inner diameter of the tubular cannula. The inner diameter of the first guide part can remain the same irrespective of the outer diameter. The expanded region at the proximal end of the first guide part may be disposed on the outside or on the outer wall of the first guide part. The expanded region may, for example, have a partial or full circumferential thickening of the wall of the first guide part to the outside. The expanded region can also have material ribs or other structures which are formed, for example, integrally with the outer wall of the first guide part. By the provision of the expanded region at the proximal end of the first guide part, the first guide part can be prevented from slipping too deeply into the cannula. The expanded region can thus function as a kind of stopper which limits or defines the maximum insertion depth of the first guide part in the cannula when the first guide part is being inserted into the cannula.

According to other embodiments of the cannula device, the first and second guide parts can each have a part of a fixing device at the proximal end, by means of which the second guide part can be fastened thereto relative to the first guide part. A relative movement of the two guide parts to one another can be prevented by means of the fixing device. The position in which the two guide parts can be fixed relative to one another by means of the fixing device can define a desired position of the two guide parts relative to one another. The desired position may correspond to the position in which the second guide part is disposed in the first guide part such that the distal ends of the two guide parts form a conically narrowing distal end, which may be substantially free of steps.

According to other embodiments of the cannula device, the fixing device can comprise a female thread arranged in the first guide part and a matching male thread arranged on the second guide part. To relatively fix the two guide parts to each other, the two guide parts can be screwed together, wherein in the screwed-together position, the two guide parts can be arranged relative to one another in the desired position.

According to other embodiments of the cannula device, when the modular cannula device is in an assembled state, it may have an overall tapering distal end formed at least by the tapering ends of the first and second guide parts. The diameter of the tapering end may decrease gradually from its largest circumference to its smallest circumference so that the tapering end of the cannula device is basically step-free. The tubular cannula itself may have a blunt end, wherein the edge region may be beveled such that there is an almost constant transition between the outer wall of the cannula and the outer wall of the first guide part.

According to other embodiments of the cannula device, a radiopaque marker can be arranged in the region of the distal end of the tubular cannula and preferably takes the form of a completely circumferential ring in the lateral surface of the cannula. A radiopaque material is understood to mean one which does not allow X-rays to pass unhindered and is thus recognizable in the x-ray image. The radiopaque marker may also comprise more than one fully circumferential ring, or may instead or additionally have other structures of radiopaque material. The use of more than one structure of radiopaque material may facilitate determining from recorded x-ray images the location of the cannula in the patient during an operation. The radiopaque marker at the distal end of the cannula can be used, for example, to represent the position of the end or exit opening of the cannula at its target location (for example in the pulmonary trunk) also over time after implantation in the patient. In particular, a radiopaque marker can be attached to the distal edge of the cannula, for example in the form of a ring of radiopaque material.

According to other embodiments of the cannula device, the distal end region of the cannula, which may preferably be at least 2 cm long and may comprise, for example, the distal 4 cm of the length of the cannula, may be made of a softer material than the remaining part of the cannula, for example silicone. The risk of injury due to perforations of blood vessels or the excessive ablation of tissue from the vascular walls can thereby be reduced. Generally, the cannula body may be made of, for example, polyethylene, Teflon, polyurethane or nylon.

According to other embodiments of the cannula device, the cannula may be wire-reinforced. For this purpose, the cannula can have a wire reinforcement which is attached to or embedded in the outer wall of the cannula and reinforces/stabilizes the cannula body. The distal end region, which may be softer, may be excluded from this. A region at the proximal end of the cannula may have a ⅜ inch (0.95 cm) connection. This region at the proximal end of the cannula may also be excluded from the wire reinforcement.

According to other embodiments of the cannula device, the cannula may be made of a softer material than the first guide part. This makes it possible to ensure that the cannula device has the necessary stiffness while being inserted into the patient. After the cannula device has been brought into its target position and then, at the end of surgery, the two-part guide part which stiffens it is taken out so that only the soft cannula remains in the patient.

According to other embodiments of the cannula device, the material can be provided with a pretensioning in at least one part in the distal region of the cannula, so that the corresponding part of the cannula has/assumes an arcuate shape without external force being applied. According to this embodiment, the cannula may be formed at least in part from a suitable shape-memory polymer. The shape-memory polymer is capable of assuming its originally provided external shape despite an intermediate strong deformation. In the present case, the interim reshaping of the external cannula is performed by the guide part inserted therein, which is formed from a stiffer material. Particularly preferably, the cannula can be provided with an native arcuate shape so that, in the final state, it adapts optimally to the shape of the right or left ventricle and does not cause discomfort to the patient while remaining in his body. The location of the region formed from the shape-memory material within the cannula may be varied to optimally adapt the position of the arcuate shape within the cannula to the patient's anatomy, which may depend on age, sex, and size. By means of an arcuate shape of the cannula, for example, the anatomy of the right ventricle and the right outflow tract of the heart can be taken into account.

The use of the modular cannula device according to the invention can make it possible to simplify and shorten the placement of a cannula. In particular, this relates to the percutaneous insertion of a cannula (for example into the pulmonary artery) using the Seldinger technique. The cannula placed by means of the cannula device described herein can be connected to a centrifugal pump without or with an oxygenator (ECMO). With a suitably selected diameter of the cannula, significantly higher flow rates can be achieved than in conventional systems. For example, the standard outer diameter of the cannula may be 21 French (F) (7 mm), but the cannulas used within the context of the invention may readily be provided in other sizes (e.g., for children or very large adults) so that the cannula may have outer diameters ranging from about 15 F (5 mm) to about 27 F (9 mm), for example in 2 F (0.66 mm) steps. The length of the cannula can also be accordingly adapted to the anatomy of the patient. For an adult, a length of the cannula within a range of 1.5 m can be assumed. The cannula of the cannula device according to the invention may have a standard connection at the proximal end, which may be ⅜ inch (0.95 cm) or ¼ inch (0.64 cm) in length.

The modular structure of the cannula device according to the invention, which is fluid-tight with respect to the relative position of the modules to each other (i.e., cannula and guide parts), provides the basis for simplifying and accelerating the process of placing a cannula, for example through the upper vena cava as an access vein. However, it should be understood that any vein may serve as an access vein, and the invention is not limited in the choice of access vein.

During passage through an access vein such as the upper vena cava, the external cannula is held rigid/straight during implantation by the guide parts. The first and second guide parts form a two-part stylet. As already described, both the first and second guide parts (i.e., the inner stylet) are open at the end so that the overall cannula device can be guided along a guide wire, for example a J-wire having a diameter of 0.035 inches (0.89 mm). If the second guide part is removed from the first guide part after insertion of the cannula device into the upper vena cava or into another large body cavity, for example, then the passage opening in the interior of the cannula device is effectively enlarged. A 0.09 inch (2.3 mm) balloon catheter can then be inserted into the cannula device, for example, and the cannula device according to the invention (with the second guide part removed) can be guided by means of the latter. In this case, the cannula can simultaneously assume the role of a sheath during implantation, and the first guide part that remains in the cannula can assume the role of a vascular dilator.

In another embodiment of the cannula device, the second guide part may be configured as a balloon catheter. As a result, the step of removing the second guide part and subsequently inserting the balloon catheter can be dispensed with, whereby this results in an additional time saving, and a further manual procedure is eliminated within the context of the overall operation.

Over the course of a typical operation, the cannula device according to the invention can be introduced by means of a guide wire (J-wire) and the subsequently used balloon catheter into the pulmonary artery via the internal jugular vein and the superior vena cava. The balloon catheter may be similar in wall thickness and surface area to a conventional, commonly used Swan-Ganz pulmonary artery catheter. Differences result from the fact that the balloon catheter used in the context of the invention can be structurally connected to a guide module. This can be accomplished, for example, by a short Luer lock thread. The balloon catheter and the cannula of the cannula device according to the invention are coordinated with one another in terms of length and diameter by printed markings and can furthermore each have radiopaque and/or echogenic markings at suitable locations in order to control the implantation and correct positioning with the aid of fluoroscopy by means of x-rays and/or ultrasound.

A single-lumen balloon catheter can particularly preferably be used in connection with the cannula device according to the invention, which allows application of contrast media that can correspondingly likewise display the end of the cannula by using length-matched modules of the cannula device. It is thus possible at any time to determine the position of the distal tip of the exterior cannula during the implantation procedure and with respect to the anatomical structures, in particular the bifurcation of the pulmonary arteries. Overall, this represents a significant improvement of previous systems.

The invention is based on the approach that the external cannula, which is the target object for implantation from the point of view of the operation, is part of the modular cannula device which is inserted into the patient at the start of the operation. Consequently, during use of the invention, the final cannula is immediately placed during the first insertion procedure. In particular, no procedures come before the placement of the cannula during which preliminary cannulas or wires are introduced into the target blood vessel and taken out again. For placing the cannula by means of the cannula device according to the invention, the cannula device is brought into contact with the vascular walls of the target vessel only once and advanced to its end position. As a result, the present invention enables considerably faster and safer implantation of a cannula compared to previously known systems.

Other advantages and embodiments of the invention will be apparent from the subsequent detailed description and the appended drawings. Exemplary embodiments of the invention, which will be described below with reference to the drawings, are schematically illustrated therein.

The figures show schematically:

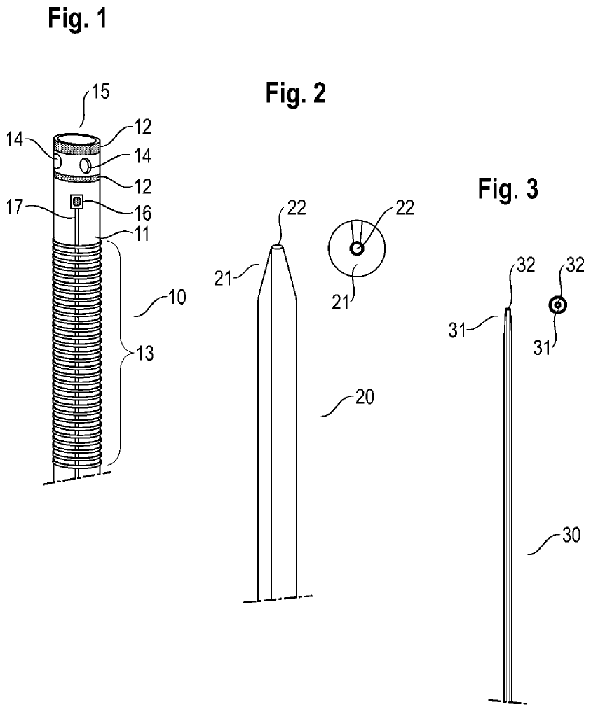

FIG. 1 a perspective side view of an exemplary embodiment of the distal end of the external cannula,

FIG. 2 a perspective side view of an exemplary embodiment of the distal end of the first guide part,

FIG. 3 a perspective side view of an exemplary embodiment of the distal end of the second guide part,

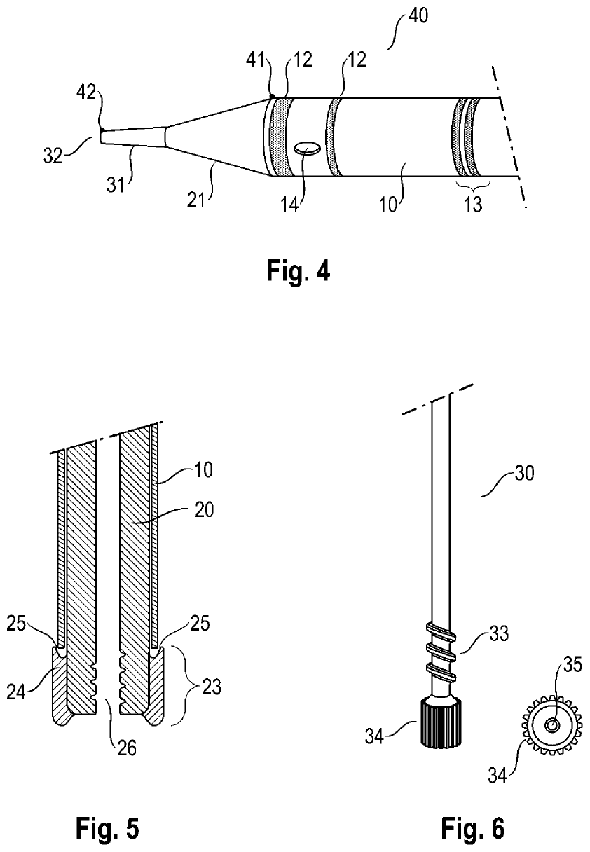

FIG. 4 a perspective side view of the distal end of the cannula device in the assembled state,

FIG. 5 a side view showing the proximal end region of the external cannula into which the first guide part is inserted,

FIG. 6 a perspective side view of the proximal end of the second guide part of the cannula device,

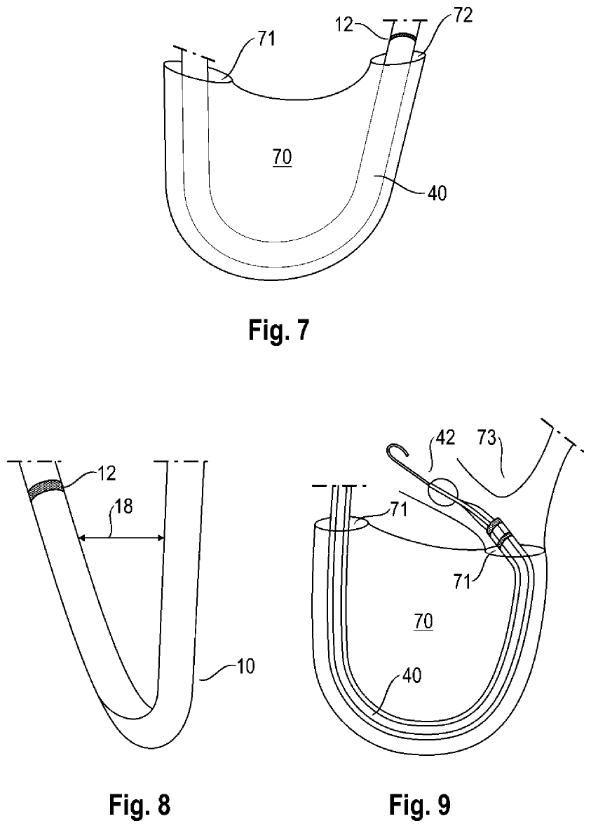

FIG. 7 a representation of an exemplary position of the cannula device in a ventricle of the heart,

FIG. 8 a diagram illustrating the pretensioning of the cannula,

FIG. 9 a diagram in which an exemplary location is illustrated of the cannula device with a balloon catheter that has been guided through during implantation.

FIG. 1 shows a perspective side view of an exemplary embodiment of the distal end of the exterior cannula of the cannula device 10. In the example shown, the cannula 10 has a cylindrical or tubular basic shape 11 and has an opening 15 at the distal end. The round cross-section of the cannula 10 may generally have a different geometric shape and may be round or oval, for example, or resemble a regular polygon (e.g., pentagon, hexagon, octagon, etc.). Rings 12 of radiopaque material are arranged in the wall of the cannula 10. One of the rings is located directly at the distal end of the cannula 10 so as to define the opening 15 in the cannula 10. The other ring 12 is arranged at a distance from the opening 15. Provided in the wall of the cannula 10 are lateral holes 14, the position, number and size of which can be selected depending on the intended use of the cannula device according to the invention. Furthermore, a coil 13 is provided around the outer wall of the cannula 10 and assumes the function of a reinforcing or stiffening structure. The material from which the coil 13 is made may preferably be stiffer than the material from which the wall of the cannula 10 is made. A distal region of the cannula 10 extending between the distal end of the coil 13 and the distal opening 15 of the cannula 10 is not reinforced/stiffened and is therefore more flexible/pliable than the part of the cannula 10 reinforced by the coil 13. Furthermore, a flow sensor 16 is mounted in the distal region of the cannula 10 by means of which a fluid flow present on the distal wall of the cannula can be examined. Data are transmitted and power is supplied to the flow sensor 16 by means of (at least) one line 17 running on the outer wall (or even within the outer wall).

FIG. 2 shows a perspective side view of an exemplary embodiment of the distal end of the first guide part 20. The first guide part 20 has a substantially cylindrical basic shape narrowing or tapering in the distal region 21 toward the distal end 22. Just as in the case of the exterior cannula (see FIG. 1), an opening is also provided here at the distal end 22. In other words, the first guide part 20 (just like the exterior cannula 10 shown in FIG. 1) is hollow in the interior. The first guide part 20 is dimensioned such that it can be inserted into the cannula 10 shown in FIG. 1. By adapting the dimensions of the two modules to one another, the contact surface between the two modules is tight, so that no fluid (in particular blood) can pass therethrough. The internal diameter of the cannula 10 and the geometric shape of its cross-section therefore correspond to the external diameter or geometric shape of the cross-section of the first guide part 20. In addition, a top view of the distal region 21 of the first guide part 20 is illustrated at top right in FIG. 2.

FIG. 3 illustrates the distal end region of the second guide part 30. The second guide part 30 has a structure that is basically similar to the first guide part 10 (while initially ignoring the smaller diameter). That is to say, the second guide part 30 also has a substantially cylindrical basic shape that narrows or tapers in the distal region 31 toward the distal end 32. The distal end 32 terminates with an opening located at the distal end of the hollow channel extending through the interior of the second guide part 30. Analogously to the configuration of the first guide part 20 with respect to the cannula 10, the second guide part 30 is dimensioned such that it can be inserted into the first guide part 20 shown in FIG. 2. The contact area between these two modules is also tight so that no fluid (in particular blood) can pass therethrough. This can be slid on a guide wire through the opening at the distal end 32 of the second guide part.

In FIG. 4, a perspective side view is shown of an exemplary embodiment of the distal end of the modular cannula device 40. In the assembled state which corresponds to the implantation state, the second guide part 30 is arranged within the first guide part 20, and the first guide part 20 is in turn arranged within the cannula 10 as the outermost module of the cannula assembly 40. In the assembled, ready-to-use state, the modules are arranged relative to one another such that the distal end region 21 of the first guide part protrudes out of the opening 15 at the distal end of the cannula 10 and, at the same time, the distal end region 31 of the second guide part protrudes out of the opening at the distal end of the first guide part 20. By means of corresponding fixing or locking mechanisms, such a configuration of the modules of the cannula assembly 40 can be fixed so that it is automatic, i.e., without a physician having to hold the modules relative to each other. That is to say, the modules in each case in contact with each other can be connected to each other in pairs so that during a surgery, this configuration is maintained and the entire cannula device 40 can be moved as a unit. A fixing or locking mechanism independent of the other mechanism can be provided in each case between two of the three components. In particular, in the assembled state, the modules can be positioned relative to one another in such a way that the distal end regions 21, 31 of the first and second guide parts 20, 30 merge into one another smoothly and “gradually”, in particular step-free or edge-free. In other words, a continuously smooth surface may result from the edge of the opening 15 at the distal end of the cannula 10 up to the opening at the distal end of the second guide part 30. As shown in FIG. 4, the slopes of the lateral surfaces of the conically narrowing distal end regions 21, 31 of the guide parts 20, 30 may be different. In particular, these production parameters can be optimized/adapted for the particular type of operation in which the cannula device according to the invention is to be used. In addition, a connecting line between a first point 41 at the edge of the opening 15 and a second point 42 at the opening at the distal end of the second guide part 30, when viewing the cannula device 40 in a longitudinal cross-section, need not necessarily be straight, but may have a curvature. For example, the connecting line between the two points 41 42 may have a concave or convex curvature.

FIG. 5 shows a side view in which the proximal end region of the external cannula 10 with a first guide part 20 inserted therein is illustrated. At its proximal end, the first guide part 20 has a thickened region 23 with an outer diameter which is greater than the diameter of the hollow channel in the interior of the cannula 10. The thickened region 23 is formed as a radial projection 24 which can project over the entire circumference or only segmentally from the outer wall of the first guide part 20. The projection 24, which lies against the edge of the proximal end of the cannula 10, prevents the first guide part 20 from being inserted too deeply into the cannula 10. In the present case, the projection 24 has an indentation 25 at its distal end (or the segments arranged along the circumference of the first guide part 20 have corresponding indentations) which receives the edge region of the proximal end of the cannula 10. The length of the first guide part 20 can be selected such that, upon the engagement of the edge region of the proximal end of the cannula 10 in the indentation 25, the first guide part 20 assumes a position opposite the cannula 10 as shown in FIG. 4. In addition, the first guide part 20 has a female thread 26 which is arranged at the proximal end of the first guide part 20.

In FIG. 6, a perspective side view of the proximal end region of the second guide part 30 of the cannula device 40 is shown. At the proximal end, a grip region 34 is provided, which is mounted upstream of a male thread 33. The male thread 33 is formed such that when the second guide part 30 is pushed into the first guide part 20, it can be screwed into the female thread 26 of the first guide part 20. The male thread 33 and the female thread 26 provide two parts of a fixing device by means of which the two guide parts can be connected to one another. At the same time, the maximum insertion depth of the second guide part 30 into the first guide part 20 can be defined. In the lower right corner of FIG. 6, an underside view of the proximal end of the second guide part 30 is additionally shown. The proximal end has an opening 35 which preferably has the same diameter as the opening at the distal end 31 of the second guide part 30.

The fixing device, designed as a threaded device by way of example, represents only one of many possibilities of how the two guide parts 20, 30 can be connected to one another. For example, a latch-turn lock may also be used. In general, the fixing device represents an optional feature which can also be omitted entirely. Instead, the grip region, which represents a thickened region in relation to the remaining part of the second guide part 30, can function as a limit for the insertion depth of the second guide part 10 into the first guide part 20. At a maximum insertion or push-in depth of the second guide part 30 into the first guide part 20, the guide parts 20, 30 can assume the position shown in FIG. 4 relative to one another.

In the representation in FIG. 7, an exemplary location is illustrated of the cannula device 40 during a percutaneous implantation process through the right ventricle 70 of the heart. As shown, the cannula device 40 extends arcuately between the tricuspid valve 71 and the pulmonary valve 72. During the operation in which the cannula device 40 is advanced through the corresponding veins and through the two heart valves 71, 72 of the right ventricle 70 into the right ventricular outflow tract, the placed guide wire or balloon catheter predefines the shape of the cannula device 40 according to the invention. In other words, when the cannula device 40 is advanced along the guide wire or balloon catheter, its shape follows the course of the guide wire. After the cannula device 40 has assumed its final position at which the external cannula 40 is to remain, the two-part guide part 20, 30 (or even only the first guide part 20 still remaining in the cannula 10) is withdrawn from the cannula 10. In this case, the cannula 10 can assume its native shape and curve, for example, as shown in FIG. 8, arcuately to conform to the anatomy of the right ventricle 70. The angle 18 between the legs of the arcuate or round V-shape may lie within a range from about 10° to about 25°, preferably within a range from about 10° to about 15°. The arcuate shape can be produced by a material pretensioning introduced during production, in that the cannula 10 is made of a shape-memory polymer at least in the relevant sub-region. As a result, the solitary cannula 10 automatically assumes the arcuate shape provided for it and exerts no or only minimal pressure on the inner wall of the heart. Discomfort to the patient during the time the cannula 10 is in place can thus be prevented or at least greatly reduced.

FIG. 9 shows a diagram illustrating an exemplary location of the cannula device 40 with a through-guided balloon catheter 42 during implantation. In the exemplary scenario shown, the second guide part 30 has been removed from the cannula device 40 and a balloon catheter 42 has been inserted instead, with which a guide path can be created out of the right ventricle 70 upward to and through the pulmonary valve 72 into the right ventricular outflow tract 73. The cannula device 40 can then be slid out of the right ventricle 70 along this guide path and into the right ventricular outflow tract 73.

In conclusion, it should be noted that the present description has only been formulated by way of example in light of the right ventricular assist device as a primary example of the use of the cannula device according to the invention. This exemplary application by no means constitutes a limitation on the intended use of the cannula device according to the invention. Rather, the use of the modular cannula device according to the invention is advantageously possible in all operations in which a cannula is to be placed in a vessel and/or organ of the patient.

1. Modular cannula device comprising:
an external tubular cannula;
a first tubular guide part that has a tapering distal end and can be inserted into the cannula so that its outer wall rests tightly against the inner wall of the cannula, and
a second tubular guide part which has a tapering, narrowing distal end and can be inserted into the first guide part so that its outer wall rests tightly against the inner wall of the first guide part.

2. Modular cannula device according to claim 1,
wherein the opening angle of the tapering distal end of the first guide part is greater than the opening angle of the tapering distal end of the second guide part.

3. Modular cannula device according to claim 1 or 2,
wherein the first tubular guide part has an enlarged region at the proximal end with an external diameter that is greater than the internal diameter of the tubular cannula.

4. Modular cannula device according to any one of claims 1 to 3,
wherein the first and second guide parts each have a part of a fixing device at the proximal end, by means of which the second guide part can be fastened to the first guide part relative thereto.

5. Modular cannula device according to claim 4,
wherein the fixing device comprises a female thread arranged in the first guide part and a matching male thread arranged on the second guide part.

6. Modular cannula device according to any one of claims 1 to 5,
wherein, when the modular cannula device is in an assembled state, it may have a tapering distal end formed at least by the tapering ends of the first and second guide parts.

7. Modular cannula device according to any one of claims 1 to 6,
wherein a radiopaque marker is arranged in the region of the distal end of the tubular cannula, which is preferably designed as a completely circumferential ring in the lateral surface of the cannula.

8. Modular cannula device according to any one of claims 1 to 7,
wherein the distal end region of the cannula, which is preferably at least 2 cm long, is made of a softer material than the remaining part of the cannula.

9. Modular cannula device according to any one of claims 1 to 8,
wherein the cannula is made of a softer material than the first guide part.

10. Modular cannula device according to any one of claims 1 to 9,
wherein the material is provided with a pretensioning in at least one part in the distal region of the cannula so that the corresponding part of the cannula has an arcuate shape without an external force being applied.
